(19) United States
(12) Patent Application Publication
(10) Pub. No.: US 2025/0255503 A1
(43) Pub. Date: Aug. 14, 2025

(54) BIOLOGICAL SIGNAL OBTAINING DEVICE, BIOLOGICAL SIGNAL OBTAINING METHOD, AND RECORDING MEDIUM

(71) Applicant: SHARP KABUSHIKI KAISHA, Osaka (JP)

(72) Inventors: RYOTA TOMIZAWA, Osaka (JP); KEN NAKASHIMA, Osaka (JP); YOSHIHISA ADACHI, Osaka (JP); AZUSA NAKANO, Osaka (JP)

(21) Appl. No.: 19/049,298

(22) Filed: Feb. 10, 2025

(30) Foreign Application Priority Data
Feb. 14, 2024 (JP) 2024-019894

(51) Int. Cl. A61B 5/024; A61B 5/00; H04N 23/88; H04N 25/10; H04N 25/78

(52) U.S. Cl. CPC A61B 5/02427; A61B 5/0077; H04N 23/88; H04N 25/10; H04N 25/78

(57) ABSTRACT

A biological signal obtaining device includes: an imaging unit that performs imaging in accordance with an imaging condition, and to output pixel values of a plurality of pixels in each of channels that are included in two or more channels each corresponding to one of two or more wavelengths different from one another; a representative value calculating unit that calculates a representative value of each of the channels from pixel values of a plurality of intra-region pixels in a region showing a biological subject, the pixel values being included in the pixel values of the plurality of pixels; a control unit that sets the imaging condition so that the representative value of each of the channels is greater than, or equal to, a set value of each of the channels; and a biological signal calculating unit that calculates a biological signal from representative values of the two or more channels.

### CROSS-REFERENCE TO RELATED APPLICATION

The present application claims priority from Japanese Application JP2024-019894, filed on Feb. 14, 2024, the content of which is hereby incorporated by reference into this application.

### BACKGROUND

### 1. Field

The present disclosure relates to a biological signal obtaining device, a biological signal obtaining method, and a recording medium.

### 2. Description of the Related Art

Japanese Unexamined Patent Application Publication No. 2021-177822 discloses an imaging control device. The imaging control device controls, for example, exposure time, gain, and white balance so that an average value μr of the distribution of R signal values, an average value μg of the distribution of G signal values, and an average value μb of the distribution of the B signal values of pixels of a skin region in a biological image satisfy conditions μr<Rmax, |μg−St|<ε, and μb>Bmin. Such control makes it possible to increase variations in a G signal value with respect to variations in intensity of G light absorbed by hemoglobin. As a result, a signal-to-noise ratio of a biological signal can be increased (see paragraphs [0010], [0034], [0082], and [0085]).

### SUMMARY

The imaging control device disclosed in Japanese Unexamined Patent Application Publication No. 2021-177822 can contain the G signal value within a range suitable for detecting a biological signal. However, the imaging control device cannot contain an R signal value or a B signal value within a range suitable for detecting a biological signal and noise. Hence, when detecting a biological signal from the G signal value, the imaging control device cannot remove noise using the R signal value and the B signal value.

The present disclosure is devised in view of the above problems. The present disclosure sets out to provide a biological signal obtaining device, a biological signal obtaining method, and a computer-readable recording medium that can obtain a biological signal reflecting a condition of a biological subject in high precision.

A biological signal obtaining device according to a first aspect of the present disclosure includes: an imaging unit that performs imaging in accordance with an imaging condition, and to output pixel values of a plurality of pixels in each of channels that are included in two or more channels each corresponding to one of two or more wavelengths different from one another; a representative value calculating unit that calculates a representative value of each of the channels from pixel values of a plurality of intra-region pixels in a region showing a biological subject, the pixel values being included in the pixel values of the plurality of pixels; a control unit that sets the imaging condition so that the representative value of each of the channels is greater than, or equal to, a set value of each of the channels; and a biological signal calculating unit that calculates a biological signal from representative values of the two or more channels.

A biological signal obtaining method according to a second aspect of the present disclosure includes: imaging in accordance with an imaging condition, and outputting pixel values of a plurality of pixels in each of channels included in two or more channels each corresponding to one of two or more wavelengths different from one another; calculating a representative value of each of the channels from pixel values of a plurality of intra-region pixels in a region showing a biological subject, the pixel values being included in the pixel values of the plurality of pixels; setting the imaging condition so that the representative value of each of the channels is greater than, or equal to, a set value of each of the channels; and calculating a biological signal from representative values of the two or more channels.

A computer-readable recording medium containing a program to cause a computer to execute: imaging, by an imaging unit, in accordance with an imaging condition, and outputting, by the imaging unit, pixel values of a plurality of pixels in each of channels included in two or more channels each corresponding to one of two or more wavelengths different from one another; calculating a representative value of each of the channels from pixel values of a plurality of intra-region pixels in a region showing a biological subject, the pixel values being included in the pixel values of the plurality of pixels; setting the imaging condition so that the representative value of each of the channels is greater than, or equal to, a set value of each of the channels; and calculating a biological signal from representative values of the two or more channels.

### BRIEF DESCRIPTION OF THE DRAWINGS

FIG. 1 is a block diagram of a pulse wave signal obtaining device according to a first embodiment;

FIG. 2 is a drawing illustrating details of processing performed by an imaging unit included in the pulse wave signal obtaining device of the first embodiment;

FIG. 3 is a drawing illustrating details of processing performed by a control unit included in the pulse wave signal obtaining device of the first embodiment;

FIG. 4 is a drawing illustrating details of processing performed by the control unit included in the pulse wave signal obtaining device of the first embodiment;

FIG. 5 is a drawing illustrating details of processing performed by a representative value calculating unit included in the pulse wave signal obtaining device of the first embodiment;

FIG. 6 is a drawing illustrating a relationship among values to be used for the processing performed by the control unit included in the pulse wave signal obtaining device of the first embodiment;

FIG. 7 is a drawing illustrating details of processing performed by the representative value calculating unit, the control unit, and a pulse wave signal calculating unit included in the pulse wave signal obtaining device of the first embodiment;

FIG. 8 is a drawing illustrating details of processing performed by the representative value calculating unit, the control unit, and the pulse wave signal calculating unit included in the pulse wave signal obtaining device of a first modification of the first embodiment;

FIG. 9 is a drawing illustrating details of processing performed preferably by the representative value calculating unit and the control unit included in the pulse wave signal obtaining device of the first embodiment;

FIG. 10 is a block diagram of an imaging unit included in the pulse wave signal obtaining device of the first embodiment;

FIG. 11 is a drawing illustrating details of processing performed by the representative value calculating unit included in the pulse wave signal obtaining device of a second modification of the first embodiment and in a pulse wave signal obtaining device of a first modification of a second embodiment;

FIG. 12 is a drawing illustrating details of processing performed by a display control unit included in the pulse wave signal obtaining device of a third modification of the first embodiment and in the pulse wave signal obtaining device of a second modification of the second embodiment;

FIG. 13 is a block diagram illustrating a computer to serve as the representative value calculating unit, the pulse wave signal calculating unit, the control unit, and the display control unit included in the pulse wave signal obtaining device of the first embodiment;

FIG. 14 is a flowchart showing a sequence of processing performed by the pulse wave signal obtaining device of the first embodiment; and

FIG. 15 is a block diagram illustrating an imaging unit included in the pulse wave signal obtaining device of the second embodiment.

### DETAILED DESCRIPTION OF THE DISCLOSURE

Embodiments of the present disclosure will be described below with reference to the drawings. Note that, throughout the drawings, like reference signs denote identical or similar constituent features. Such features will not be repeatedly elaborated upon.

### 1 First Embodiment

### 1.1 Summary of Pulse Wave Signal Obtaining Device

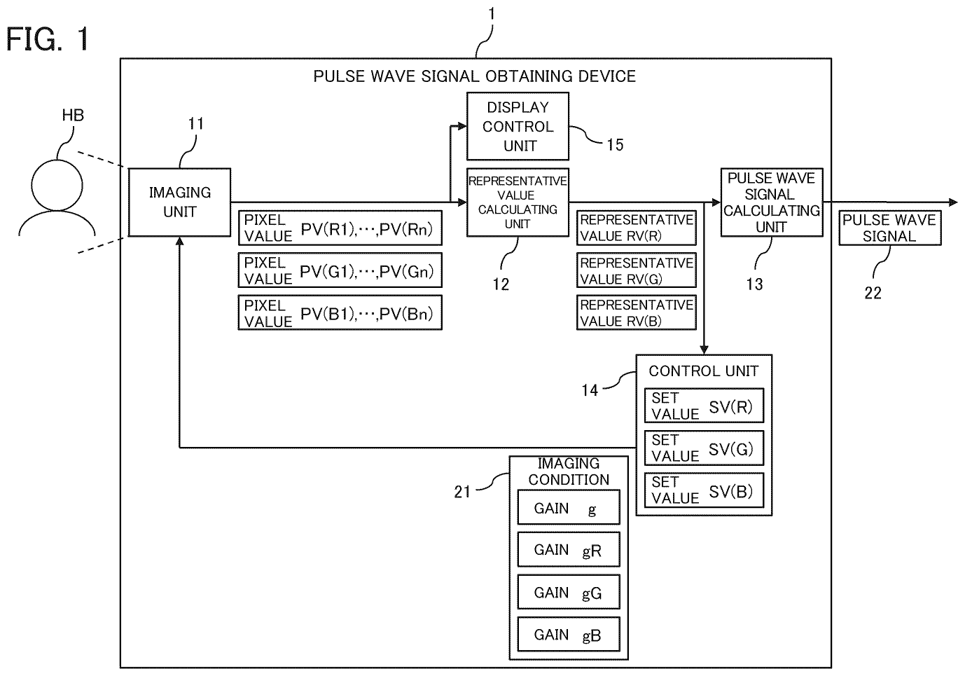

FIG. 1 is a block diagram illustrating a pulse wave signal obtaining device of a first embodiment.

A pulse wave signal obtaining device 1 of the first embodiment illustrated in FIG. 1 images a human body HB, and obtains a pulse wave signal 22 of the imaged human body HB. The pulse wave signal 22 to be obtained is a signal indicating a pulse wave. The human body HB is an example of a biological subject. The pulse wave signal obtaining device 1 may image a biological subject other than the human body HB, and obtain the pulse wave signal 22 of the imaged biological subject. The pulse wave signal 22 is an example of a biological signal. A configuration to be described below may be adopted to a biological signal obtaining device that obtains a biological signal other than the pulse wave signal 22. The biological signal to be obtained reflects a state of the biological subject.

As illustrated in FIG. 1, the pulse wave signal obtaining device 1 includes: an imaging unit 11; a representative value calculating unit 12; a pulse wave signal calculating unit 13; a control unit 14; and a display control unit 15.

The imaging unit 11 performs imaging in accordance with an imaging condition 21, and outputs a moving image. The moving image to be output includes a plurality of frame images.

The imaging unit 11 is an RGB camera. Hence, each of the frame images included in the plurality of frame images includes: pixel values PV(R1), . . . , PV(Rn) of a plurality of pixels R1, . . . , Rn for an R (red) channel; pixel values PV(G1), . . . , PV(Gn) of a plurality of pixels G1, . . . , Gn for a G (green) channel; and pixel values PV(B1), . . . , PV(Bn) of a plurality of pixels B1, . . . , Bn for a B (blue) channel.

The R channel, the G channel, and the B channel respectively corresponds to an R wavelength band, a G wavelength band, and a B wavelength band.

Three wavelength bands of the R wavelength band, the G wavelength band, and the B wavelength band are an example of two or more wavelength bands different from one another. Three channels of the R channel, the G channel, and the B channel are an example of two or more channels different from one another. The frame image may include pixel values of a plurality of pixels in each of channels that are included in two or more channels each corresponding to one of two or more wavelength bands other than the three wavelength bands.

The representative value calculating unit 12 calculates a representative value RV (R) of the R channel from pixel values of a plurality of intra-region pixels in an interest region. Here, the pixel values are included in the output pixel values PV(R1), . . . , PV(Rn). The representative value calculating unit 12 calculates a representative value RV(G) of the G channel from pixel values of a plurality of intra-region pixels in the interest region. Here, the pixel values are included in the output pixel values PV(G1), . . . , PV(Gn). The representative value calculating unit 12 calculates a representative value RV(B) of the B channel from pixel values of a plurality of intra-region pixels in the interest region. Here, the pixel values are included in the output pixel values PV(B1), . . . , PV(Bn). The representative values RV(R), RV(G), and RV(B) to be calculated include an average value, a maximum value, a minimum value, a mode value, and a median value.

The control unit 14 sets the imaging condition 21 so that the representative value RV(R) is greater than, or equal to, a set value SV(R) of the R channel, the representative value RV(G) is greater than, or equal to, a set value SV(G) of the G channel, and the representative value RV(B) is greater than, or equal to, a set value SV(B) of the B channel.

The pulse wave signal calculating unit 13 calculates the pulse wave signal 22 from the representative values RV(R), RV(G), and RV(B), using a technique such as independent component analysis or pigment component separation. The pulse wave signal 22 to be calculated reflects a pulse wave of the human body HB.

The display control unit 15 causes a display to present a frame image corresponding to the output pixel values PV(R1), . . . , PV(Rn), PV(G1), . . . , PV(Gn), and PV(B1), . . . , PV(Bn).

### 1.2 Quantization of Light-Reception Amount

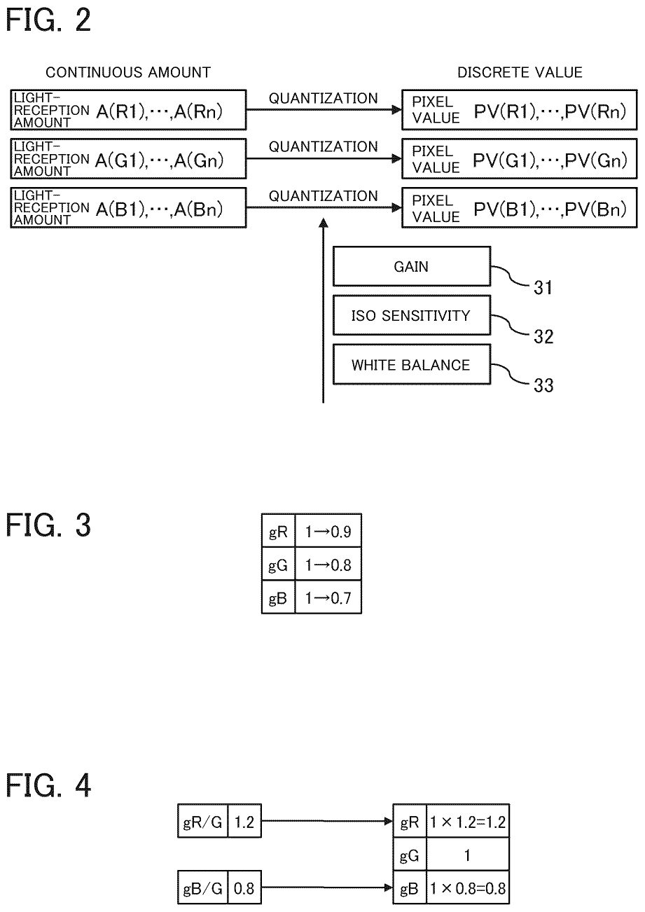

FIG. 2 is a drawing illustrating details of processing performed by the imaging unit included in the pulse wave signal obtaining device of the first embodiment.

As illustrated in FIG. 2, the imaging unit 11 converts light-reception amounts A(R1), . . . , A(Rn) of light received with the pixels R1, . . . , Rn into the pixel values PV(R1), . . . , PV(Rn). The imaging unit 11 converts light-reception amounts A(G1), . . . , A(Gn) of light received with the pixels G1, . . . , Gn into the pixel values PV(G1), . . . , PV(Gn). The imaging unit 11 converts light-reception amounts A(B1), . . . , A(Bn) of light received with the pixels B1, . . . , Bn into the pixel values PV(B1), . . . , PV(Bn).

The imaging unit 11 increases the pixel values PV(R1), . . . , PV(Rn) as the light-reception amounts A(R1), . . . , A(Rn) increase. The imaging unit 11 increases the pixel values PV(G1), . . . , PV(Gn) as the light-reception amounts A(G1), . . . , A(Gn) increase. The imaging unit 11 increases the pixel values PV(B1), . . . , PV(Bn) as the light-reception amounts A(B1), . . . , A(Bn) increase.

The light-reception amounts A(R1), . . . , A(Rn), A(G1), . . . , A(Gn), and A(B1), . . . , A(Bn) are continuous amounts. The pixel values PV(R1), . . . , PV(Rn), PV(G1), . . . , PV(Gn), and PV(B1), . . . , PV(Bn) are discrete values. Hence, the imaging unit 11 quantizes the light-reception amounts A(R1), . . . , A(Rn) to obtain the pixel values PV(R1), . . . , PV(Rn). The imaging unit 11 quantizes the light-reception amounts A(G1), . . . , A(Gn) to obtain the pixel values PV(G1), . . . , PV(Gn). The imaging unit 11 quantizes the light-reception amounts A(B1), . . . , A(Bn) to obtain the pixel values PV(B1), . . . , PV(Bn).

### 1.3 Parameters to be Used for Control of Imaging Unit

As illustrated in FIG. 2, parameters to be used for control of the imaging unit 11 include a gain 31, an ISO sensitivity 32, and a white balance 33.

The gain 31 and the ISO sensitivity 32 indicate a degree of amplification to be performed on pixel signals indicating the light-reception amounts A(R1), . . . , A(Rn), A(G1), . . . , A(Gn), and A(B1), . . . , A(Bn). Hence, if the light-reception amounts A(R1), . . . , A(Rn), A(G1), . . . , A(Gn), and A(B1), . . . , A(Bn) are constant, the pixel values PV(R1), . . . , PV(Rn), PV(G1), . . . , PV(Gn), and PV(B1), . . . , PV(Bn) increase as the gain 31 or the ISO sensitivity 32 increases.

The white balance 33 indicates a ratio of pixel values in three channels including: pixel values indicating the light-reception amounts A(R1), . . . , A(Rn); pixel values indicating the light-reception amounts A(G1), . . . , A(Gn); and pixel values indicating the light-reception amounts A(B1), . . . , A(Bn).

### 1.4 Imaging Condition

As illustrated in FIG. 1, the imaging condition 21 includes: a gain g; a gain gR of the R channel; a gain gG of the G channel; and a gain gB of the B channel.

The imaging unit 11 outputs the pixel values PV(R1), . . . , PV(Rn), PV(G1), . . . , PV(Gn), and PV(B1), . . . , PV(Bn) corresponding to the gain g. If the gain g increases, the imaging unit 11 increases all of the pixel values PV(R1), . . . , PV(Rn), PV(G1), . . . , PV(Gn), and PV(B1), . . . , PV(Bn).

The imaging unit 11 outputs the pixel values PV(R1), . . . , PV(Rn) corresponding to the gain gR. If the gain gR increases, the imaging unit 11 increases the pixel value PV(R1), . . . , PV(Rn) without changing any of the pixel values PV(G1), . . . , PV(Gn) or PV(B1), . . . , PV(Bn).

The imaging unit 11 outputs the pixel values PV(G1), . . . , PV(Gn) corresponding to the gain gG. If the gain gG increases, the imaging unit 11 increases the pixel value PV(G1), . . . , PV(Gn) without changing any of the pixel values PV(R1), . . . , PV(Rn) or PV(B1), . . . , PV(Bn).

The imaging unit 11 outputs the pixel values PV(B1), . . . , PV(Bn) corresponding to the gain gB. If the gain gB increases, the imaging unit 11 increases the pixel value PV(B1), . . . , PV(Bn) without changing any of the pixel values PV(R1), . . . , PV(Rn) or PV(G1), . . . , PV(Gn).

When adjusting the gain 31 or the ISO sensitivity 32, the control unit 14 adjusts the gain g in order to adjust the pixel values PV(R1), . . . , PV(Rn), PV(G1), . . . , PV(Gn), and PV(B1), . . . , PV(Bn) as a whole. When the gain g is 1, if pixel values PV(Ri), PV(Gi), and PV(Bi) are 100, the pixel values PV(Ri), pixel values PV(Ri), PV(Gi), and PV(Bi) are 150 when the gain g is 1.5.

When correcting the white balance 33, the control unit 14 individually adjusts the gains gR, gG, and gB, in order to individually adjust the pixel values PV(R1), . . . , PV(Rn), the pixel values PV(G1), . . . , PV(Gn), and the pixel values PV(B1), . . . , PV(Bn). The control unit 14 can adjust a gain included in each of the gains gR, gG, and gB independently of remaining gains included in the gains gR, gG, and gB. Thanks to such a feature, the control unit 14 can adjust a ratio of the pixel values in three channels including the pixel values PV(R1), . . . , PV(Rn), the pixel values PV(G1), . . . , PV(Gn), and pixel values PV(B1), . . . , PV(Bn). When the white balance 33 is not corrected, and the gains gR, gG, and gB are 1, if the pixel values PV(Ri), PV(Gi), and PV(Bi) are 100, the pixel values PV(Ri), PV(Gi), and PV(Bi) are respectively 90, 80, and 70 when the white balance is corrected and the gains gR, gG, and gB are respectively 0.9, 0,8, and 0.7.

### 1.5 Setting Gains

FIGS. 3 and 4 are drawings illustrating details of processing performed by the control unit included in the pulse wave signal obtaining device of the first embodiment.

When correcting the white balance 33, the control unit 14 changes the gains gR, gG, and gB from a reference gain of each of the R channel, the G channel, and the B channel. For example, as illustrated in FIG. 3, the control unit 14 changes the gains gR, gG, and gB from the reference gains 1 of the R channel, the G channel, and the B channel, to 0.9, 0.8, and 0.7.

Alternatively, when correcting the white balance 33, the control unit 14 sets a ratio of a gain of a second channel to a gain of a first channel. The control unit 14 sets the gain of the first channel to the reference gain of the first channel, and sets the gain of the second channel to a product of the reference gain of the first channel and the set ratio. For example, as illustrated in FIG. 4, the control unit 14 sets the G channel as the first channel, and the R channel and the B channel as the second channels. The control unit 14 sets a ratio gR/G of the gain gR to the gain gG to 1.2, and a ratio gB/G of the gain gB to the gain gG to 0.8. The control unit 14 sets: the gain gG to 1; that is, the reference gain of the G channel; the gain gR to 1.2; that is, a product of the reference gain 1 of the G channel and the ratio gR/G=1.2; and the gain gB to 0.8; that is, a product of the reference gain 1 of the G channel and the ratio gB/G =0.8.

### 1.6 Calculating Representative Values

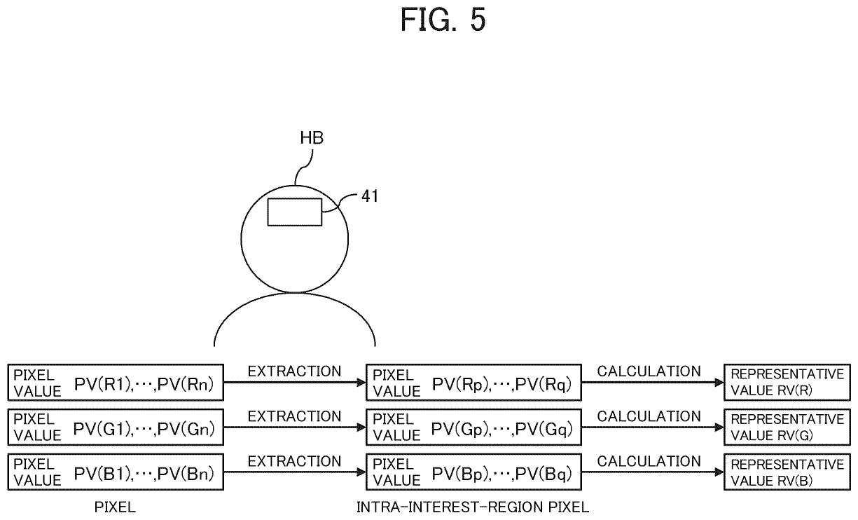

FIG. 5 is a drawing illustrating details of processing performed by the representative value calculating unit included in the pulse wave signal obtaining device of the first embodiment.

As illustrated in FIG. 5, the representative value calculating unit 12 extracts, from the pixel values PV(R1), . . . , PV(Rn), pixel values PV(Rp), . . . , PV(Rq) of intra-interest-region pixels Rp, . . . , Rq included in an interest region 41 that is set. The representative value calculating unit 12 extracts, from the pixel values PV(G1), . . . , PV(Gn), pixel values PV(Gp), . . . , PV(Gq) of intra-interest-region pixels Gp, . . . , Gq included in the interest region 41 that is set. The representative value calculating unit 12 extracts, from the pixel values PV(B1), . . . , PV(Bn), pixel values PV(Bp), . . . , PV(Bq) of intra-interest-region pixels Bp, . . . , Bq included in the interest region 41 that is set.

The interest region 41 is a region included in a frame image and showing the human body HB. A temporal change in the color of the interest region 41 reflects a pulse wave of the human body HB. Hence, from the pixel values PV(Rp), . . . , PV(Rq), PV(Gp), . . . , PV(Gq), and PV(Bp), . . . , PV(Bq), the pulse wave signal 22 reflecting the pulse wave of the human body HB can be calculated.

The interest region 41 is set inside an image of a part of exposed skin of the human body HB. For example, the interest region 41 is set in an image of a face, a cheek, a forehead, a palm, a wrist, or a sole.

The representative value calculating unit 12 calculates a representative value RV(R) of the extracted pixel values PV(Rp), . . . , PV(Rq). The representative value calculating unit 12 calculates a representative value RV(G) of the extracted pixel values PV(Gp), . . . , PV(Gq). The representative value calculating unit 12 calculates a representative value RV(B) of the extracted pixel values PV(Bp), . . . , PV(Bq).

### 1.7 Advantage of Setting Imaging Condition to Raise Representative Value Higher than, or Equal to, Set Value

Light incident on the skin of the human body HB is mainly absorbed by melanin. Hence, when the light is diffused and reflected on the skin of the human body HB, the light amount of the light in each of the wavelength bands decreases as melanin exhibits higher absorption intensity in absorbing the light in each wavelength band. Melanin exhibits larger absorption intensity for a light R, a light G, and a light B in the stated order. Hence, when a white light is incident on the skin of the human body HB, in the light diffused and reflected on the skin of the human body HB, the light amount is smaller in the order of the light R, the light G, and the light B.

The light incident on the skin of the human body HB is also absorbed by hemoglobin. Hence, the light amount of the light diffused and reflected on the skin of the human body HB decreases as the amount of hemoglobin increases. Hence, the light amount of light, in each wavelength band, included in the light diffused and reflected on the skin of the human body HB temporarily varies in synchronization with a temporal variation in the amount of hemoglobin by pulsation of blood vessels. Among the light amount of the light R, the light amount of the light G, and the light amount of the light B, the light amount of the light G shows the most significant temporal variation. However, the temporal variation in the light amount of the light G is also minute. Hence, suppose a case where the imaging unit 11 is a general-purpose camera, and a bit length of the pixel values PV(R1), . . . , PV(Rn), PV(G1), . . . , PV(Gn), and PV(B1), . . . , PV(Bn) is short; that is, for example, the bit length is 8 bits and the pixel values PV(R1), . . . , PV(Rn), PV(G1), . . . , PV(Gn), and PV(B1), . . . , PV(Bn) cannot take any values other than 256 levels of values from 0 to 255. In order to detect a small temporal variation in the light amount of light in each wavelength band because of a temporal variation in the amount of hemoglobin, a large number of levels need to be assigned to the temporal variations in the pixel values PV(R1), . . . , PV(Rn), PV(G1), . . . , PV(Gn), and PV(B1), . . . , PV(Bn).

In order to assign a large number of levels to the temporal variations in the pixel values PV(R1), . . . , PV(Rn), PV(G1), . . . , PV(Gn), and PV(B1), . . . , PV(Bn), it is effective to increase the pixel values PV(R1), . . . , PV(Rn), PV(G1), . . . , PV(Gn), and PV(B1), . . . , PV(Bn) as long as the pixel values PV(R1), . . . , PV(Rn), PV(G1), . . . , PV(Gn), and PV(B1), . . . , PV(Bn) are not saturated. This can be understood from the fact as follows. For example, suppose a range of a minute temporal variation in the light amount of the light G caused by a minute temporal variation in the amount of hemoglobin is approximately 1% of a steady light amount of the light G. If a pixel value PV(Gi) is 100, the number of levels to be assigned to a temporal variation in the pixel value PV(Gi) is approximately 1. If the pixel value PV(Gi) is 200, the number of levels is approximately 2.

Considering the above fact, the control unit 14 sets the imaging condition 21 so that the representative value RV(R) is greater than, or equal to, the set value SV(R), the representative value RV(G) is greater than, or equal to, the set value SV(G), and the representative value RV(B) is greater than, or equal to, the set value SV(B). The settings can increase the number of levels to be assigned to the temporal variations in the representative values RV(R), RV(G), and RV(B). Such a feature can precisely reflect in the pulse wave signal 22 the temporal variation in the light amount of the light, in each of the wavelength bands, included in the light diffused and reflected on the skin of the human body HB.

### 1.8 Range of Set Value

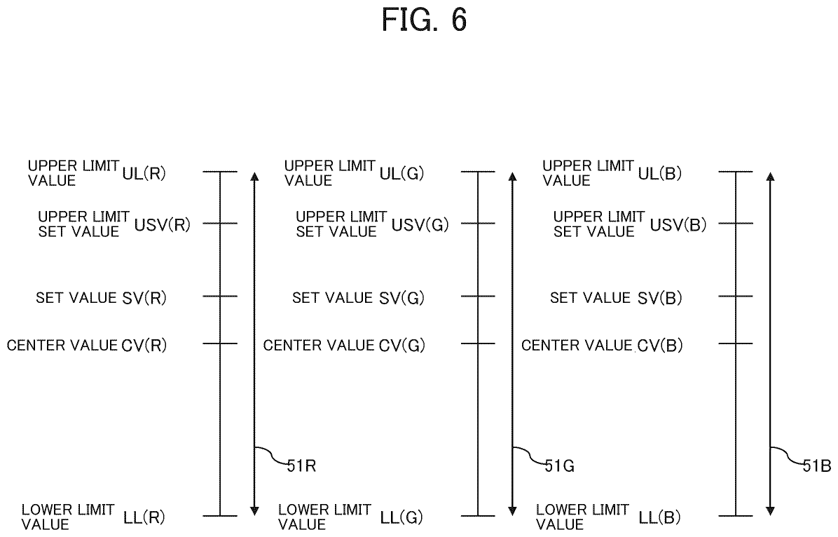

FIG. 6 is a drawing illustrating a relationship among values to be used for the processing performed by the control unit included in the pulse wave signal obtaining device of the first embodiment.

As illustrated in FIG. 6, the pixel values PV(R1), . . . , PV(Rn) can take values within a range 51R from a lower limit value LL(R) of the R channel to an upper limit value UL(R) of the R channel. The pixel values PV(G1), . . . , PV(Gn) can take values within a range 51G from a lower limit value LL(G) of the G channel to an upper limit value UL(G) of the G channel. The pixel values PV(B1), . . . , PV(Bn) can take values within a range 51B from a lower limit value LL(B) of the B channel to an upper limit value UL(B) of the B channel.

The set value SV(R) is desirably greater than, or equal to, a center value CV(R) of the range 51R, and is set as large as possible. The set value SV(G) is desirably greater than, or equal to, a center value CV(G) of the range 51G, and is set as large as possible. The set value SV(B) is desirably greater than, or equal to, a center value CV(B) of the range 51B, and is set as large as possible. The settings can increase the representative values RV(R), RV(G), and RV(B), thereby successfully increasing the number of levels to be assigned to the temporal variations in the representative values RV(R), RV(G), and RV(B).

The set value SV(R), the set value SV(G), and the set value SV(B) may be either the same value or different values.

The control unit 14 may set the imaging condition 21 so that: the representative value RV(R) is greater than, or equal to, the set value SV(R), and smaller than, or equal to, an upper limit set value USV(R) of the R channel; the representative value RV(G) is greater than, or equal to, the set value SV(G), and smaller than, or equal to, an upper limit set value USV(G) of the G channel; and the representative value RV(B) is greater than, or equal to, the set value SV(B), and smaller than, or equal to, an upper limit set value USV(B) of the B channel. That is, the control unit 14 may set the imaging condition 21 so that: the representative value RV(R) takes a value within a range from the set value SV(R) to the upper limit set value USV(R); the representative value RV(G) takes a value within a range from the set value SV(G) to the upper limit set value USV(G); and the representative value RV(B) takes a value within a range from the set value SV(B) to the upper limit set value USV(B).

The upper limit set values USV(R), USV(G), and USV(B) may be either the same value or different values.

The upper limit set value USV(R) is smaller than the upper limit value UL(R) of the range 51R. The upper limit set value USV(G) is smaller than the upper limit value UL(G) of the range 51G. The upper limit set value USV(B) is smaller than the upper limit value UL(B) of the range 51B. Such features can reduce saturation of the pixel values PV(R1), . . . , PV(Rn), PV(G1), . . . , PV(Gn), and PV(B1), . . . , PV(Bn) even if the pixel values PV(R1), . . . , PV(Rn), PV(G1), . . . , PV(Gn), and PV(B1), . . . , PV(Bn) increase because of, for example, motion of the human body HB and a rise in intensity of illumination light illuminating the human body HB.

When a bit length of the pixel values PV(R1), . . . , PV(Rn), PV(G1), . . . , PV(Gn), and PV(B1), . . . , PV(Bn) is 8 bits, for example, the set values SV(R), SV(G), and SV(B) are 200, and the upper limit set values USV(R), USV(G), and USV(B) are 230. Hence, the representative values RV(R), RV(G), and RV(B) take values within the range of 200 to 230.

A size of the range from the set value SV(R) to the upper limit set value USV(R) is determined to be the same as a size of the range of the variations in the pixel values PV(R1), . . . , PV(Rn) because of, for example, motion of the human body HB. A size of the range from the set value SV(G) to the upper limit set value USV(G) is determined to be the same as a size of the range of the variations in the pixel values PV(G1), . . . , PV(Rn) because of, for example, motion of the human body HB. A size of the range from the set value SV(B) to the upper limit set value USV(B) is determined to be the same as a size of the range of the variations in the pixel values PV(B1), . . . , PV(Bn) because of, for example, motion of the human body HB. Such features have an advantageous effect: when the range from the set value SV(G) to the upper limit set value USV(G), the range from the set value SV(B) to the upper limit set value USV(B), and the range from the set value SV(G) to the upper limit set value USV(G) are excessively narrow such that a required time period for setting the imaging condition 21 is excessively long, these features can reduce the time period.

### 1.9 Calculating Representative Value from Pulse Wave Signal

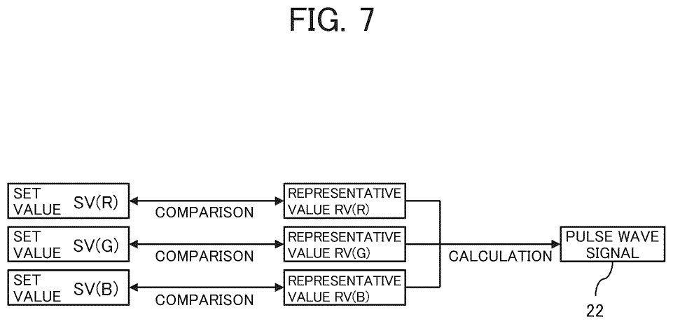

FIG. 7 is a drawing illustrating details of processing performed by the representative value calculating unit, the control unit, and the pulse wave signal calculating unit included in the pulse wave signal obtaining device of the first embodiment.

In the first embodiment, as illustrated in FIG. 7, the representative value calculating unit 12 calculates the representative values RV(R), RV(G), and RV(B). Furthermore, the control unit 14 respectively compares the representative values RV(R), RV(G), and RV(B) with the set values SV(R), SV(G), and SV(B), and sets the imaging condition 21 so that the representative value RV(R) is greater than, or equal to, the set value SV(R), the representative value RV(G) is greater than, or equal to, the set value SV(G), and the representative value RV(B) is greater than, or equal to, the set value SV(B). Moreover, the pulse wave signal calculating unit 13 calculates the pulse wave signal 22 from the temporal variations in the representative values RV(R), RV(G), and RV(B) calculated after the imaging condition 21 is set.

The temporal variation in each of the representative values RV(R), RV(G), and RV(B) includes: a pulse wave component due to a temporal variation in the amount of hemoglobin by pulsation of the human body HB; a first noise component due to a temporal variation in the amount of melamine; and a second noise component due to motion of the human body HB. Differences are observed between: a ratio of the pulse wave component to the first noise component to the second noise component included in the temporal variation in the representative value RV(R); a ratio of the pulse wave component to the first noise component to the second noise component included in the temporal variation in the representative value RV(G); and a ratio of the pulse wave component to the first noise component to the second noise component included in the temporal variation in the representative value RV(B).

The pulse wave signal calculating unit 13 calculates the pulse wave signal 22 from the temporal variations in the representative values in three channels including the representative values RV(R), RV(G), and RV(B), thereby successfully separating from one another the pulse wave component, the first noise component, and the second noise component included in the temporal variation in each of the representative values RV(R), RV(G), and RV(B). Hence, using the temporal variations in the representative value RV(R) and the representative value RV(B), the pulse wave signal calculating unit 13 can remove the first noise component and the second noise component from the temporal variation in the representative value RV(G), extract the pulse wave component from the temporal variation in the representative value RV(G), and calculate the pulse wave signal 22 from the extracted pulse wave component.

As to the pulse wave signal obtaining device 1, a large number of levels are assigned to the temporal variations in the representative values RV(R), RV(G), and RV(B). Such a feature can precisely reflect the pulse waves in the representative values RV(R), RV(G), and RV(B). Hence, the feature can precisely reflect the pulse waves in the pulse wave signal 22 calculated from the representative values RV(R), RV(G), and RV(B).

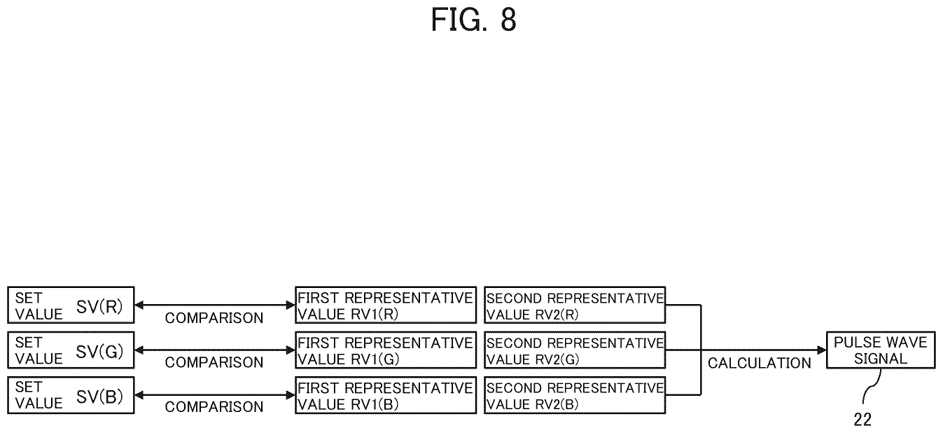

FIG. 8 is a drawing illustrating details of processing performed by the representative value calculating unit, the control unit, and the pulse wave signal calculating unit included in the pulse wave signal obtaining device of a first modification of the first embodiment.

In the first modification of the first embodiment, as illustrated in FIG. 8, the representative value calculating unit 12 can calculate, as the representative value RV(R), a first representative value RV1(R) and a second representative value RV2(R). Furthermore, the representative value calculating unit 12 can calculate, as the representative value RV(G), a first representative value RV1(G) and a second representative value RV2(G). Moreover, the representative value calculating unit 12 can calculate, as the representative value RV(B), a first representative value RV1(B) and a second representative value RV2(B). In addition, the control unit 14 respectively compares the first representative value RV1(R), the first representative value RV1(G), and the first representative value RV1(B) with the set value SV(R), the set value SV(G), and the set value SV(B), and sets the imaging condition 21 so that the first representative value RV1(R) is greater than, or equal to, the set value SV(R), the first representative value RV1(G) is greater than, or equal to, the set value SV(G), and the first representative value RV1(B) is greater than, or equal to, the set value SV(B). Furthermore, the pulse wave signal calculating unit 13 calculates the pulse wave signal 22 from the temporal variations in the second representative value RV2(R), the second representative value RV2(G), and the second representative value RV2(B) calculated after the imaging condition 21 has been set.

The first representative value RV1(R) and the second representative value RV2(R) are two kinds of representative values calculated from the same pixel values PV(R1), . . . , PV(Rn). The first representative value RV1(G) and the second representative value RV2(G) are two kinds of representative values calculated from the same pixel values PV(G1), . . . , PV(Gn). The first representative value RV1(B) and the second representative value RV2(B) are two kinds of representative values calculated from the same pixel values PV(B1), . . . , PV(Bn). The first representative values RV1(R), RV1(G), and RV1(B) are, for example, highest values. The first representative values RV2(R), RV2(G), and RV2(B) are, for example, average values.

### 1.10 Correction of White Balance to Approximate Representative Values

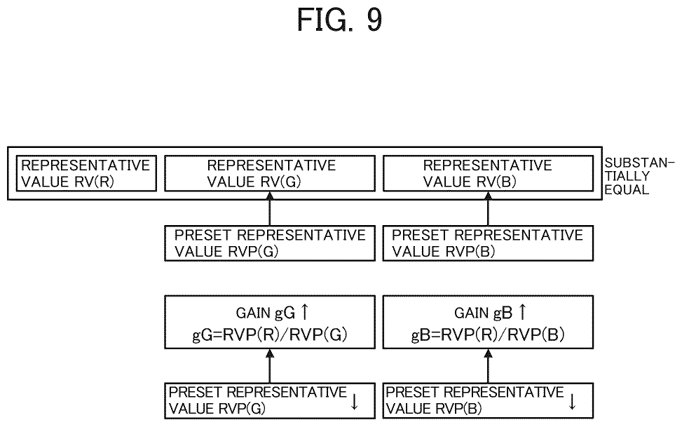

FIG. 9 is a drawing illustrating details of processing performed preferably by the representative value calculating unit and the control unit included in the pulse wave signal calculating device of the first embodiment.

In the light diffused and reflected on the skin of the human body HB, the light amount of the light R is greater than the light amount of the light G and the light amount of the light B. If the skin is illuminated with warm illumination light having strong redness, the light amount of the light R is further increased compared with the case where the skin is illuminated with white illumination light. Hence, when the imaging condition 21 is set so that the pixel values PV(R1), . . . , PV(Rn) are simply not saturated, the pixel values PV(G1), . . . , PV(Gn) and PV(B1), . . . , PV(Bn) could be excessively small. That is why a signal-to-noise ratio(S/N ratio) of the pulse wave signal 22 could be low.

Thus, as illustrated in FIG. 9, when correcting the white balance 33, the control unit 14 increases the gain gG as a preset representative value RVP(G) of the G channel becomes smaller. The preset representative value RVP(G) has been calculated by the representative value calculating unit 12 before the gain gG is set. Furthermore, when correcting the white balance 33, the control unit 14 increases the gain gB as a preset representative value RVP(B) of the B channel becomes smaller. The preset representative value RVP(B) has been calculated by the representative value calculating unit 12 before the gain gB is set. For example, the control unit 14 sets the gain gR to 1, sets the gain gG to RVP(R)/RVP(G); that is, a ratio of the preset representative value RVP(R) to the preset representative value RVP(G), and sets the gain gB to RVP(R)/RVP(B); that is, a ratio of the preset representative value RVP(R) to the preset representative value RVP(B). Thus, the control unit 14 sets the imaging conditions 21 so that the representative values RV(G) and RV(B) approximate to the representative value RV(R). Such a feature can reduce saturation of the pixel values PV(R1), . . . , PV(Rn), PV(G1), . . . , PV(Gn), and PV(B1), . . . , PV(Bn) and increase the representative values RV(R), RV(G), and RV(B). The feature can increase the number of levels to be assigned to the temporal variations in the representative values RV(R), RV(G), and RV(B). The feature can precisely reflect a pulse wave in the representative values RV(R), RV(G), and RV(B). The feature can precisely reflect a pulse wave in the pulse wave signal 22 calculated from the representative values RV(R), RV(G), and RV(B).

### 1.11 Quantization by Imaging Unit

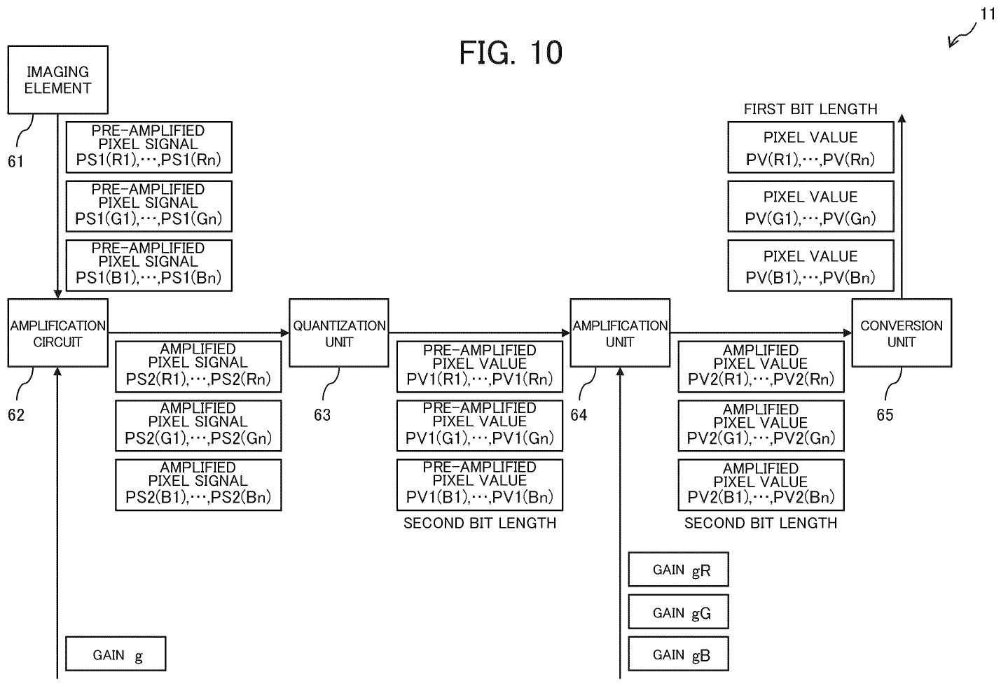

FIG. 10 is a block diagram of the imaging unit included in the pulse wave signal obtaining device of the first embodiment.

As illustrated in FIG. 10, the imaging unit 11 includes: an imaging element 61; an amplification circuit 62; a quantization unit 63; an amplification unit 64; and a conversion unit 65.

The imaging element 61 outputs pre-amplified pixel signals PS1(R1), . . . , PS1(Rn) of the pixels R1, . . . , Rn, pre-amplified pixel signals PS1(G1), . . . , PS1(Gn) of the pixels G1, . . . , Gn, and pre-amplified pixel signals PS1(B1), . . . , PS1(Bn) of the pixels B1, . . . , Bn. The output pre-amplified pixel signals PS1(R1), . . . , PS1(Rn), PS1(G1), . . . , PS1(Gn), and PS1(B1), . . . , PS1(Bn) are analog signals, and indicate continuous amounts. The imaging element 61 increases the pre-amplified pixel signals PS1(R1), . . . , PS1(Rn) as the light-reception amounts A(R1), . . . , A(Rn) increase. The imaging element 61 increases the pre-amplified pixel signals PS1(G1), . . . , PS1(Gn) as the light-reception amounts A(G1), . . . , A(Gn) increase. The imaging element 61 increases the pre-amplified pixel signals PS1(B1), . . . , PS1(Bn) as the light-reception amounts A(B1), . . . , A(Bn) increase. The imaging element 61 is, for example, a complementary metal oxide semiconductor(CMOS) image sensor, or a charge coupled device(CCD) image sensor.

The amplification circuit 62 amplifies the output pre-amplified pixel signals PS1(R1), . . . , PS1(Rn) with the gain g, and outputs amplified pixel signals PS2(R1), . . . , PS2(Rn). The amplification circuit 62 amplifies the output pre-amplified pixel signals PS1(G1), . . . , PS1(Gn) with the gain g, and outputs amplified pixel signals PS2(G1), . . . , PS2(Gn). The amplification circuit 62 amplifies the output pre-amplified pixel signals PS1(G1), . . . , PS1(Gn) with the gain g, and outputs amplified pixel signals PS2(G1), . . . , PS2(Gn). The output amplified pixel signals PS2(R1), . . . , PS2(Rn), PS2(G1), . . . , PS2(Gn), and PS2(B1), . . . , PS2(Bn) are analog signals, and indicate continuous amounts. The amplification circuit 62 increases the amplified pixel signals PS2(R1), . . . , PS2(Rn), PS2(G1), . . . , PS2(Gn), and PS2(B1), . . . , PS2(Bn) as the gain g increases. The amplification circuit 62 increases the amplified pixel signal PS2(R1), . . . , PS2(Rn) as the pre-amplified pixel signal PS1(R1), . . . , PS1(Rn) increase. The amplification circuit 62 increases the amplified pixel signal PS2(G1), . . . , PS2(Gn) as the pre-amplified pixel signal PS1(G1), . . . , PS1(Gn) increase. The amplification circuit 62 increases the amplified pixel signal PS2(B1), . . . , PS2(Bn) as the pre-amplified pixel signal PS1(B1), . . . , PS1(Bn) increase.

The imaging element 61 and the amplification circuit 62 constitute a pixel signal output unit that outputs the amplified pixel signal to be quantized, namely PS2(R1), . . . , PS2(Rn), PS2(G1), . . . , PS2(Gn), and PS2(B1), . . . , PS2(Bn).

The quantization unit 63 quantizes the output amplified pixel signals PS2(R1), . . . , PS2(Rn), and outputs pre-amplified pixel values PV1(R1), . . . , PV1(Rn). The quantization unit 63 quantizes the output amplified pixel signals PS2(G1), . . . , PS2(Gn), and outputs pre-amplified pixel values PV1(G1), . . . , PV1(Gn). The quantization unit 63 quantizes the output amplified pixel signals PS2(B1), . . . , PS2(Bn), and outputs pre-amplified pixel values PV1(B1), . . . , PV1(Bn). The pre-amplified pixel values PV1(R1), . . . , PV1(Rn), PV1(G1), . . . , PV1(Gn), and PV1(B1), . . . , PV1(Bn), which are indicated by digital signals, take discrete values, and have a second bit length. The quantization unit 63 increases the pre-amplified pixel values PV1(R1), . . . , PS1(Rn) as the amplified pixel signals PS2(R1), . . . , PS2(Rn) increase. The quantization unit 63 increases the pre-amplified pixel values PV1(G1), . . . , PS1(Gn) as the amplified pixel signals PS2(G1), . . . , PS2(Gn) increase. The quantization unit 63 increases the pre-amplified pixel values PV1(B1), . . . , PS1(Bn) as the amplified pixel signals PS2(B1), . . . , PS2(Bn) increase.

The amplification unit 64 amplifies the output pre-amplified pixel values PV1(R1), . . . , PV1(Rn) with the gain gR, and outputs amplified pixel values PV2(R1), . . . , PV2(Rn). The amplification unit 64 amplifies the output pre-amplified pixel values PV1(G1), . . . , PV1(Gn) with the gain gG, and outputs amplified pixel values PV2(G1), . . . , PV2(Gn). The amplification unit 64 amplifies the output pre-amplified pixel values PV1(B1), . . . , PV1(Bn) with the gain gB, and outputs amplified pixel values PV2(B1), . . . , PV2(Bn). The amplified pixel value PV2(R1), . . . , PV2(Rn), PV2(G1), . . . , PV2(Gn), and PV2(B1), . . . , PV2(Bn), which are indicated by digital signals, take discrete values and have the second bit length. The amplification unit 64 increases the amplified pixel values PV2(R1), . . . , PV2(Rn) as the gain gR increases. The amplification unit 64 increases the amplified pixel values PV2(G1), . . . , PV2(Gn) as the gain gG increases. The amplification unit 64 increases the amplified pixel values PV2(B1), . . . , PV2(Bn) as the gain gB increases. The amplification unit 64 increases the amplified pixel values PV2(R1), . . . , PV2(Rn) as the pre-amplified pixel values PV1(R1), . . . , PV1(Rn) increase. The amplification unit 64 increases the amplified pixel values PV2(G1), . . . , PV2(Gn) as the pre-amplified pixel values PV1(G1), . . . , PV1(Gn) increase. The amplification unit 64 increases the amplified pixel values PV2(B1), . . . , PV2(Bn) as the pre-amplified pixel values PV1(B1), . . . , PV1(Bn) increase.

The conversion unit 65 converts the output amplified pixel values PV2(R1), . . . , PV2(Rn) into the pixel values PV(R1), . . . , PV(Rn). The conversion unit 65 converts the output amplified pixel values PV2(G1), . . . , PV2(Gn) into the pixel values PV(G1), . . . , PV(Gn). The conversion unit 65 converts the output amplified pixel values PV2(B1), . . . , PV2(Bn) into the pixel values PV(B1), . . . , PV(Bn). The pixel values PV(R1), . . . , PV(Rn), PV(G1), . . . , PV(Gn), and PV(B1), . . . , PV(Bn), which are indicated by digital signals, take discrete values, and have a first bit length. The second bit length is longer than the first bit length. Hence, the conversion unit 65 reduces a bit depth of a pixel value from the second bit length to the first bit length.

The quantization unit 63, the amplification unit 64, and the conversion unit 65 constitute, as a whole, a quantization unit that quantizes the amplified pixel signals PS2(R1), . . . , PS2(Rn), PS2(G1), . . . , PS2(Gn), and PS2(B1), . . . , PS2(Bn), and converts the quantized signals into the pixel values PV(R1), . . . , PV(Rn), PV(G1), . . . , PV(Gn), and PV(B1), . . . , PV(Bn).

### 1.12 Reducing Loss of Information Related to Pulse Wave

As to the imaging unit 11, the gain g, which is used for adjusting the gain 31 and the ISO sensitivity 32, is applied to the pre-amplified pixel signals to be obtained before the quantization, namely PS1(R1), . . . , PS1(Rn), PS1(G1), . . . , PS1(Gn), and PS1(B1), . . . , PS1(Bn). Furthermore, the gains gR, gG, and gB, which are used for adjusting the white balance 33, are applied to the pre-amplified pixel values to be obtained after the quantization, namely PV1(R1), . . . , PV1(Rn), PV1(G1), . . . , PV1(Gn), and PV1(B1), . . . , PV1(Bn). In this case, when the R channel is focused, if the gain gR is not an integer, the pre-amplified pixel values PV1(R1), . . . , PV1(Rn) are multiplied by the gain gR that is not an integer. Thus, the amplified pixel values PV2(R1), . . . , PV2(Rn) are obtained. Hence, an error could be produced such that the amplified pixel value PV2(Ri) might deviate from the product of the pre-amplified pixel value PV1(Ri) and the gain gR. For example, when the pre-amplified pixel value PV1(Ri) is 3 and the gain gR is 0.5, the product of the pre-amplified pixel value PV1(Ri) and the gain gR; that is, 3×0.5=1.5, is rounded to an integer 2. As a result, the amplified pixel value PV2(Ri) is obtained. Hence, an error is produced such that the amplified pixel value PV2(Ri) deviates from the product of the pre-amplified pixel value PV1(Ri) and the gain gR. Furthermore, when the pre-amplified pixel value PV1(Ri) is 4 and the gain gR is 0.5, the product of the pre-amplified pixel value PV1(Ri) and the gain gR; that is, 4×0.5=2, is the amplified pixel value PV2(Ri). That is, in either case where the pre-amplified pixel value PV1(Ri) is 3 or where the pre-amplified pixel value PV1(Ri) is 4, the amplified pixel value PV2(Ri) is 2. This means that a portion of the information included in the pre-amplified pixel value PV1(Ri) and related to a pulse wave is lost in the amplified pixel value PV2(Ri). Furthermore, when the pre-amplified pixel value PV1(Ri) is 4 and the gain gR is 1.2, the product of the pre-amplified pixel value PV1(Ri) and the gain gR; that is, 4×1.2=4.8, is rounded to an integer 5. As a result, the amplified pixel value PV2(Ri) is obtained. Hence, an error is produced such that the amplified pixel value PV2(Ri) deviates from the product of the pre-amplified pixel value PV1(Ri) and the gain gR. The same applies to the case where either the G channel or the B channel is focused.

The amplified pixel values PV2(G1), . . . , PV2(Gn) are obtained from the pre-amplified pixel values PV1(G1), . . . , PV1(Gn) including abundant information related to a pulse wave. The control unit 14 desirably sets the gain gG to an integer in order to avoid a loss of a portion of the information. For example, the control unit 14 sets the gain gR to RVP(G)/RVP(R), and sets the gains gG and gB to 1. Thus, the control unit 14 corrects the white balance 33, so that the representative values RV(R) and RV(G) are substantially equal to each other and the representative value RV(B) is smaller than the representative values RV(R) and RV(G). The control unit 14 then adjusts the gain 31 and the ISO sensitivity 32, so that the pixel values PV(R1), . . . , PV(Rn), PV(G1), . . . , PV(Gn), and PV(B1), . . . , PV(Bn) increase without being saturated. Such a feature can precisely reflect, in the pulse wave signal 22, the abundant information included in the pixel values PV(G1), . . . , PV(Gn) and related to a pulse wave.

### 1.13 Estimation of Pixel Value before Correction of White Balance

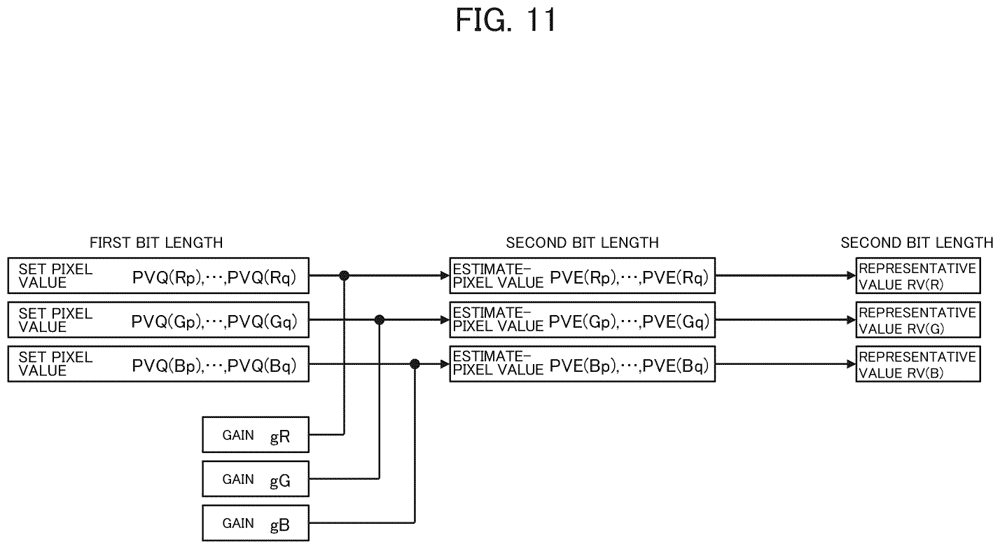

FIG. 11 is a drawing illustrating details of processing performed by the representative value calculating unit included in the pulse wave signal obtaining device of a second modification of the first embodiment.

In the second modification, as illustrated in FIG. 11, the representative value calculating unit 12 estimates estimate-pixel values PVE(Rp), . . . , PVE(Rq) from the gain gR and the set pixel values PVQ(Rp), . . . , PVQ(Rq) output by the imaging unit 11 after the gain gR has been set. Furthermore, the representative value calculating unit 12 estimates estimate-pixel values PVE(Gp), . . . , PVE(Gq) from the gain gG and set pixel values PVQ(Gp), . . . , PVQ(Gq) output by the imaging unit 11 after the gain gG has been set. Moreover, the representative value calculating unit 12 estimates estimate-pixel values PVE(Bp), . . . , PVE(Bq) from the gain gB and set pixel values PVQ(Bp), . . . , PVQ(Bq) output by the imaging unit 11 after the gain gB has been set. The estimate-pixel values PVE(Rp), . . . , PVE(Rq) are estimate values indicating magnitudes of the amplified pixel signals PS2(Rp), . . . , PS2(Rq), and are estimate values of the pre-amplified pixel values PV1(Rp), . . . , PV1(Rq). The estimate-pixel values PVE(Gp), . . . , PVE(Gq) are estimate values indicating magnitudes of the amplified pixel signals PS2(Gp), . . . , PS2(Gq), and are estimate values of the pre-amplified pixel values PV1(Gp), . . . , PV1(Gq). The estimate-pixel values PVE(Bp), . . . , PVE(Bq) are estimate values indicating magnitudes of the amplified pixel signals PS2(Bp), . . . , PS2(Bq), and are estimate values of the pre-amplified pixel values PV1(Bp), . . . , PV1(Bq). The pixel values PV(Rp), . . . , PV(Rq), PV(Gp), . . . , PV(Gq), and PV(Bp), . . . , PV(Bq) have the first bit length. The estimate-pixel values PVE(Rp), . . . , PVE(Rq), PVE(Gp), . . . , PVE(Gq), and PVE(Bp), . . . , PVE(Bq) have the second bit length longer than the first bit length. The representative value calculating unit 12 utilizes, for example, super-resolution processing to estimate the estimate-pixel values PVE(Rp), . . . , PVE(Rq), PVE(Gp), . . . , PVE(Gq), and PVE(Bp), . . . , PVE(Bq) having the second bit length from the set pixel values PVQ(Rp), . . . , PVQ(Rq), PVQ(Gp), . . . , PVQ(Gq), and PVQ(Bp), . . . , PVQ(Bq) having the first bit length. Unlike the imaging unit 11, the representative value calculating unit 12 can precisely handle pixel values having a long bit length. The representative value calculating unit 12 processes a pixel value having the second bit length longer than the first bit length, thereby successfully reducing a loss of a portion of the information related to a pulse wave.

The representative value calculating unit 12 calculates the representative value RV(R) from the estimated estimate-pixel values PVE(Rp), . . . , PVE(Rq). The representative value calculating unit 12 calculates the representative value RV(G) from the estimated estimate-pixel values PVE(Gp), . . . , PVE(Gq). The representative value calculating unit 12 calculates the representative value RV(B) from the estimated estimate-pixel values PVE(Bp), . . . , PVE(Bq).

Thus, the pulse wave signal 22 can be obtained from the estimate-pixel values PVE(Rp), . . . , PVE(Rq), PVE(Gp), . . . , PVE(Gq), and PVE(Bp), . . . , PVE(Bq) having an RGB ratio reflecting the actual color of the human body HB before the white balance 33 is corrected. Hence, the obtained pulse wave signal 22 successfully reflects the pulse wave highly precisely at a scale substantially equal to the scale before the white balance 33 is corrected.

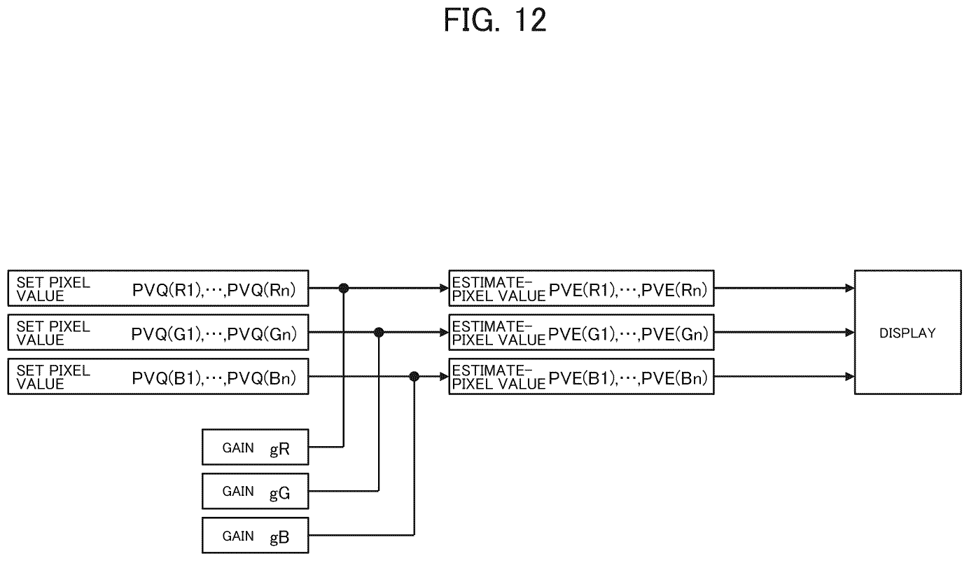

FIG. 12 is a drawing illustrating details of processing performed by the display control unit included in the pulse wave signal obtaining device of a third modification of the first embodiment.

In the third modification, as illustrated in FIG. 12, the display control unit 15 estimates the estimate-pixel values PVE(R1), . . . , PVE(Rn) from the gain gR and the set pixel values PVQ(R1), . . . , PVQ(Rn) output by the imaging unit 11 after the gain gR has been set. Furthermore, the display control unit 15 estimates the estimate-pixel values PVE(G1), . . . , PVE(Gn) from the gain gG and the set pixel values PVQ(G1), . . . , PVQ(Gn) output by the imaging unit 11 after the gain gG has been set. Moreover, the display control unit 15 estimates the estimate-pixel values PVE(B1), . . . , PVE(Bn) from the gain gB and the set pixel values PVQ(B1), . . . , PVQ(Bn) output by the imaging unit 11 after the gain gB has been set. The estimate-pixel values PVE(R1), . . . , PVE(Rn) are estimate values having magnitudes of the amplified pixel signals PS2(R1), . . . , PS2(Rn), and are estimate values of the pre-amplified pixel values PV1(R1), . . . , PV1(Rn). The estimate-pixel values PVE(G1), . . . , PVE(Gn) are estimate values indicating magnitudes of the amplified pixel signals PS2(G1), . . . , PS2(Gn), and are estimate values of the pre-amplified pixel values PV1(G1), . . . , PV1(Gn). The estimate-pixel values PVE(B1), . . . , PVE(Bn) are estimate values indicating magnitudes of the amplified pixel signals PS2(B1), . . . , PS2(Bn), and are estimate values of the pre-amplified pixel values PV1(B1), . . . , PV1(Bn).

The display control unit 15 causes a display to present a frame image corresponding to the estimated estimate-pixel values PVE(R1), . . . , PVE(Rn), PVE(G1), . . . , PVE(Gn), and PVE(B1), . . . , PVE(Bn). Such a feature makes it possible to present on the display a natural moving image having an RGB ratio reflecting the actual color of the human body HB before the white balance 33 is corrected.

### 1.14 Hardware

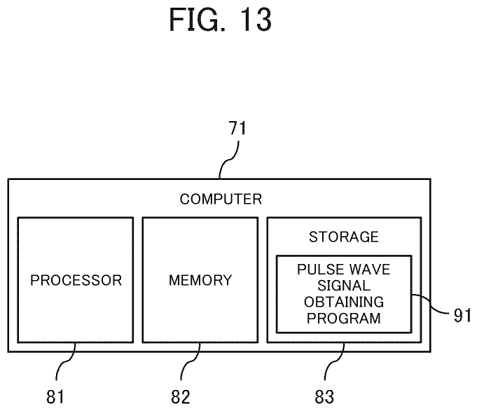

FIG. 13 is a block diagram illustrating a computer to serve as the representative value calculating unit, the pulse wave signal calculating unit, the control unit, and the display control unit included in the pulse wave signal obtaining device of the first embodiment.

As illustrated in FIG. 13, a computer 71 includes: a processor 81; a memory 82; and a storage 83. In the storage 83, a pulse wave signal obtaining program 91 is installed.

The processor 81 is, for example, a central processing unit(CPU) and a graphics processing unit(GPU). The memory 82 is, for example, a random access memory(RAM) and a read-only memory(ROM). The storage 83 is, for example, a solid-state drive(SSD), a hard disk drive(HDD), and a flash memory.

The processor 81 executes the pulse wave signal obtaining program 91 loaded from the storage 83 onto the memory 82, and causes the computer 71 to operate as the representative value calculating unit 12, the pulse wave signal calculating unit 13, the control unit 14, and the display control unit 15. Some or all of the processing executed by the computer 71 may be executed by a dedicated electronic circuit.

The pulse wave signal obtaining program 91 is received through a network, and recorded on the storage 83 serving as an internal recording medium. Alternatively, the pulse wave signal obtaining program 91 is read from an external recording medium such as an optical disk, a magnetic disk, or a flash memory device, and is recorded on the storage 83 serving as an internal recording medium.

### 1.15 Sequence of Processing

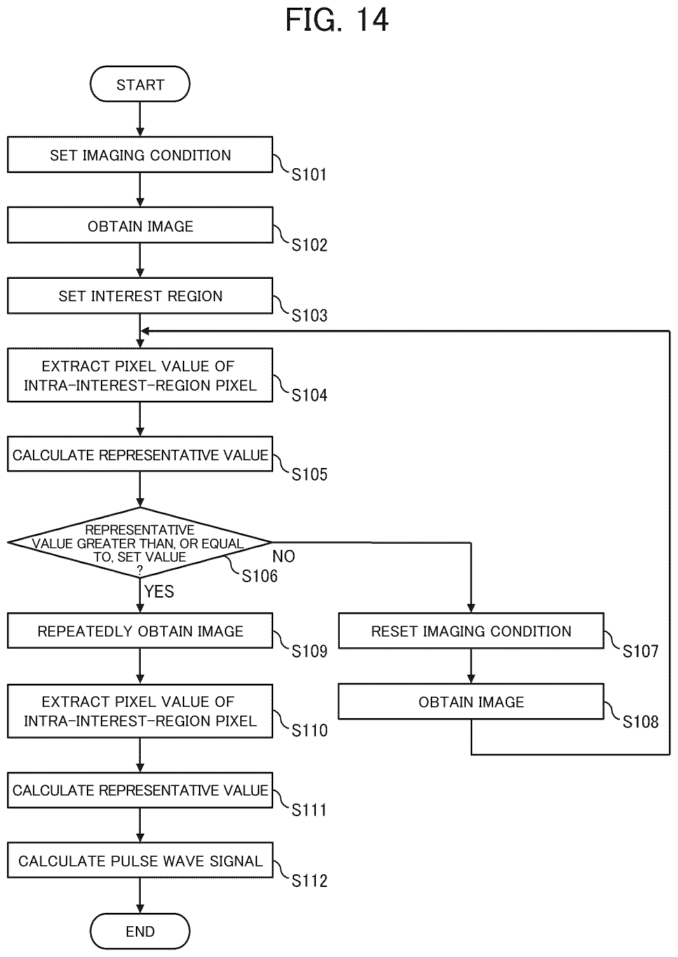

FIG. 14 is a flowchart showing a sequence of processing performed by the pulse wave signal obtaining device of the first embodiment.

The pulse wave signal obtaining device 1 executes Steps S101 to S112 shown in FIG. 14. The pulse wave signal obtaining device 1 sets the imaging condition 21 between Steps S101 and S108, and calculates the pulse wave signal 22 between Steps S109 and S112.

At Step S101, the control unit 14 sets the imaging condition 21. Here, the control unit 14 sets the imaging condition 21 to a default imaging condition. In the default imaging condition, the gains g, gR, gG, and gB are the reference gain 1.

Subsequently, at Step S102, the control unit 14 causes the imaging unit 11 to obtain an image. Thus, the imaging unit 11 performs imaging, and outputs a frame image. The frame image to be output includes the pixel values PV(R1), . . . , PV(Rn), PV(G1), . . . , PV(Gn), and PV(B1), . . . , PV(Bn).

Subsequently, at Step S103, the representative value calculating unit 12 sets the interest region 41.

Subsequently, at Step S104, the representative value calculating unit 12 extracts the pixel values PV(Rp), . . . , PV(Rq), PV(Gp), . . . , PV(Gq), and PV(Bp), . . . , PV(Bq) of the intra-interest-region pixels Rp, . . . , Rq, Gp, . . . , Gq, and Bp, . . . , Bq from the pixel values PV(R1), . . . , PV(Rn), PV(G1), . . . , PV(Gn), and PV(B1), . . . , PV(Bn) of the pixels R1, . . . , Rn, G1, . . . , Gn, and B1, . . . . Bn. Subsequently, at Step S105, the representative value calculating unit 12 calculates the representative values RV(R), RV(G), and RV(B) from the pixel values PV(Rp), . . . , PV(Rq), PV(Gp), . . . , PV(Gq), and PV(Bp), . . . , PV(Bq).

Subsequently, at Step S106, the control unit 14 determines whether the representative values RV(R), RV(G), and RV(B) are respectively greater than, or equal to, the set values SV(R), SV(G), and SV(B). If the representative values RV(R), RV(G), and RV(B) are respectively determined not to be greater than, or equal to, the set values SV(R), SV(G), and SV(B), Step S107 is executed. If the representative values RV(R), RV(G), and RV(B) are respectively determined to be greater than, or equal to, the set values SV(R), SV(G), and SV(B), Step S109 is executed.

At Step S107, the control unit 14 resets the imaging condition 21. Here, the control unit 14 resets the imaging condition 21 to an imaging condition in which the representative values RV(R), RV(G), and RV(B) are respectively expected to be greater than, or equal to, the set values SV(R), SV(G), and SV(B).

At Step S108, the control unit 14 causes the imaging unit 11 to obtain an image. Thus, the imaging unit 11 performs imaging, and outputs a frame image. The frame image to be output includes the pixel values PV(R1), . . . , PV(Rn), PV(G1), . . . , PV(Gn), and PV(B1), . . . , PV(Bn).

After Step S108 has been executed, Step S104 is executed again.

Between Step S101 and S108, the reset of the imaging condition 21 continues before the representative values RV(R), RV(G), and RV(B) are respectively greater than, or equal to, the set values SV(R), SV(G), and SV(B). After the representative values RV(R), RV(G), and RV(B) have become respectively greater than, or equal to, the set values SV(R), SV(G), and SV(B), Steps S109 to S112 are executed.

At Step S109, the control unit 14 causes the imaging unit 11 to repeatedly obtain an image. Thus, the imaging unit 11 repeatedly performs imaging, and outputs a plurality of frame images. Each of the plurality of frame images to be output includes the pixel values PV(R1), . . . , PV(Rn), PV(G1), . . . , PV(Gn), and PV(B1), . . . , PV(Bn).

Subsequently, at Step S110, the representative value calculating unit 12 extracts, for each of the plurality of frame images, the pixel values PV(Rp), . . . , PV(Rq), PV(Gp), . . . , PV(Gq), and PV(Bp), . . . , PV(Bq) of the intra-interest-region pixels Rp, . . . , Rq, Gp, . . . , Gq, and Bp, . . . , Bq from the pixel values PV(R1), . . . , PV(Rn), PV(G1), . . . , PV(Gn), and PV(B1), . . . , PV(Bn) of the pixels R1, . . . , Rn, G1, . . . , Gn, and B1, . . . . Bn.

Subsequently, at Step S111, the representative value calculating unit 12 calculates, for each of the plurality of frame images, the representative values RV(R), RV(G), and RV(B) from the pixel values PV(Rp), . . . , PV(Rq), PV(Gp), . . . , PV(Gq), and PV(Bp), . . . , PV(Bq). Hence, the representative value calculating unit 12 calculates temporal variations in the representative values RV(B), RV(G), and RV(B).

Subsequently, at Step S112, the pulse wave signal calculating unit 13 calculates the pulse wave signal 22 from the temporal variations in the representative values RV(R), RV(G), and RV(B).

### 2 Second Embodiment

Described below will be how a second embodiment is different from the first embodiment. Otherwise, the same configurations as those employed in the first embodiment are also employed in the second embodiment.

### 2.1 Quantization by Imaging Unit

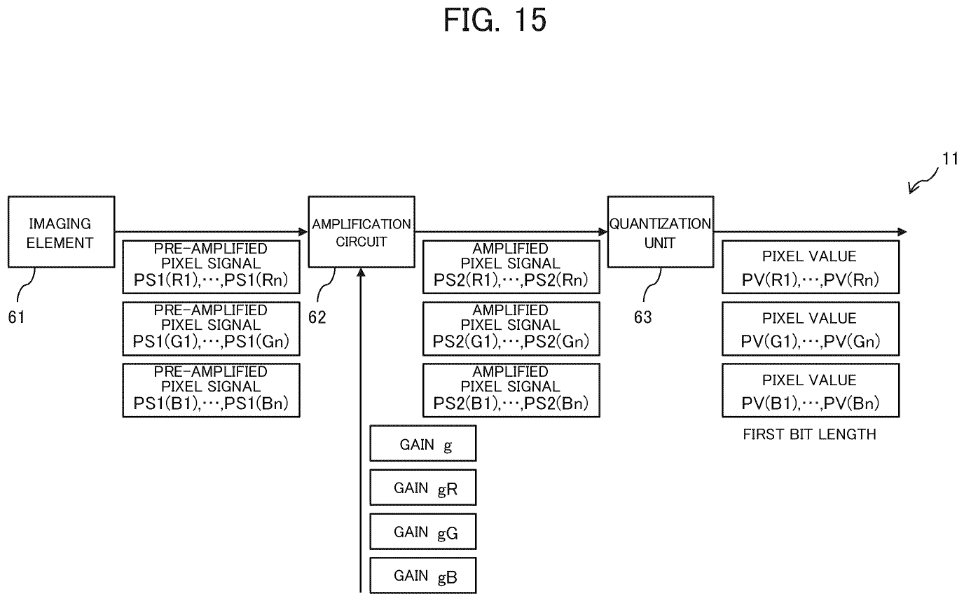

FIG. 15 is a block diagram of the imaging unit included in the pulse wave signal obtaining device of the second embodiment.

As illustrated in FIG. 15, the imaging unit 11 includes: the imaging element 61; the amplification circuit 62; and the quantization unit 63.

The imaging element 61 outputs the pre-amplified pixel signals PS1(R1), . . . , PS1(Rn) of the pixels R1, . . . , Rn, the pre-amplified pixel signals PS1(G1), . . . , PS1(Gn) of the pixels G1, . . . , Gn, and the pre-amplified pixel signals PS1(B1), . . . , PS1(Bn) of the pixels B1, . . . , Bn.

The imaging element 61 is a pixel signal output unit that outputs the pre-amplified pixel signals to be amplified, namely PS1(R1), . . . , PS1(Rn), PS1(G1), . . . , PS1(Gn), and PS1(B1), . . . , PS1(Bn).

The amplification circuit 62 amplifies the output pre-amplified pixel signals PS1(R1), . . . , PS1(Rn) with the gains g and gR, and outputs amplified pixel signals PS2(R1), . . . , PS2(Rn). The amplification circuit 62 amplifies the output pre-amplified pixel signals PS1(G1), . . . , PS1(Gn) with the gains g and gG, and outputs amplified pixel signals PS2(G1), . . . , PS2(Gn). The amplification circuit 62 amplifies the output pre-amplified pixel signals PS1(B1), . . . , PS1(Bn) with the gains g and gB, and outputs amplified pixel signals PS2(B1), . . . , PS2(Bn).

The quantization unit 63 quantizes the output amplified pixel signals PS2(R1), . . . , PS2(Rn), and outputs the pixel values PV(R1), . . . , PV(Rn). The quantization unit 63 quantizes the output amplified pixel signals PS2(G1), . . . , PS2(Gn), and outputs the pixel values PV(G1), . . . , PV(Gn). The quantization unit 63 quantizes the output amplified pixel signals PS2(B1), . . . , PS2(Bn), and outputs the pixel values PV(B1), . . . , PV(Bn).

### 2.2 Estimation of Pixel Value before Correction of White Balance

FIG. 11 is also a drawing illustrating details of processing performed by the representative value calculating unit included in the pulse wave signal obtaining device of a first modification of the second embodiment.

In the first modification, as illustrated in FIG. 11, the representative value calculating unit 12 estimates the estimate-pixel values PVE(Rp), . . . , PVE(Rq) from the gain gR and the set pixel values PVQ(Rp), . . . , PVQ(Rq) output by the imaging unit 11 after the gain gR has been set. Furthermore, the representative value calculating unit 12 estimates the estimate-pixel values PVE(Gp), . . . , PVE(Gq) from the gain gG and the set pixel values PVQ(Gp), . . . , PVQ(Gq) output by the imaging unit 11 after the gain gG has been set. Moreover, the representative value calculating unit 12 estimates the estimate-pixel values PVE(Bp), . . . , PVE(Bq) from the gain gB and the set pixel values PVQ(Bp), . . . , PVQ(Bq) output by the imaging unit 11 after the gain gB has been set. The estimate-pixel values PVE(Rp), . . . , PVE(Rq) respectively indicate magnitudes of preset pixel signals PS2(Rp), . . . , PS2(Rq) output by the amplification circuit 62 before the gain gR is set. The estimate-pixel values PVE(Gp), . . . , PVE(Gq) respectively indicate magnitudes of preset pixel signals PS2(Gp), . . . , PS2(Gq) output by the amplification circuit 62 before the gain gG is set. The estimate-pixel values PVE(Bp), . . . , PVE(Bq) respectively indicate magnitudes of preset pixel signals PS2(Bp), . . . , PS2(Bq) output by the amplification circuit 62 before the gain gB is set. The pixel values PV(Rp), . . . , PV(Rq), PV(Gp), . . . , PV(Gq), and PV(Bp), . . . , PV(Bq) have the first bit length. The estimate-pixel values PVE(Rp), . . . , PVE(Rq), PVE(Gp), . . . , PVE(Gq), and PVE(Bp), . . . , PVE(Bq) have the second bit length longer than the first bit length. The representative value calculating unit 12 utilizes, for example, super-resolution processing to estimate the estimate-pixel values PVE(Rp), . . . , PVE(Rq), PVE(Gp), . . . , PVE(Gq), and PVE(Bp), . . . , PVE(Bq) having the second bit length from the set pixel values PVQ(Rp), . . . , PVQ(Rq), PVQ(Gp), . . . , PVQ(Gq), and PVQ(Bp), . . . , PVQ(Bq) having the first bit length.

The representative value calculating unit 12 calculates the representative value RV(R) from the estimated estimate-pixel values PVE(Rp), . . . , PVE(Rq). The representative value calculating unit 12 calculates the representative value RV(G) from the estimated estimate-pixel values PVE(Gp), . . . , PVE(Gq). The representative value calculating unit 12 calculates the representative value RV(B) from the estimated estimate-pixel values PVE(Bp), . . . , PVE(Bq).

Thus, the pulse wave signal 22 can be obtained from the estimate-pixel values PVE(Rp), . . . , PVE(Rq), PVE(Gp), . . . , PVE(Gq), and PVE(Bp), . . . , PVE(Bq) having an RGB ratio reflecting the actual color of the human body HB before the white balance 33 is corrected. Hence, the obtained pulse wave signal 22 successfully reflects the pulse wave highly precisely at a scale substantially equal to the scale before the white balance 33 is corrected.

FIG. 12 is a drawing illustrating details of processing performed by the display control unit included in the pulse wave signal obtaining device of a second modification of the second embodiment.

In the second modification, as illustrated in FIG. 12, the display control unit 15 estimates the estimate-pixel values PVE(R1), . . . , PVE(Rn) from the gain gR and the set pixel values PVQ(R1), . . . , PVQ(Rn) output by the imaging unit 11 after the gain gR has been set. Furthermore, the display control unit 15 estimates the estimate-pixel values PVE(G1), . . . , PVE(Gn) from the gain gG and the set pixel values PVQ(G1), . . . , PVQ(Gn) output by the imaging unit 11 after the gain gG has been set. Moreover, the display control unit 15 estimates the estimate-pixel values PVE(B1), . . . , PVE(Bn) from the gain gB and the set pixel values PVQ(B1), . . . , PVQ(Bn) output by the imaging unit 11 after the gain gB has been set. The estimate-pixel values PVE(R1), . . . , PVE(Rn) respectively indicate magnitudes of the preset pixel signals PS2(R1), . . . , PS2(Rn) output by the amplification circuit 62 before the gain gR is set. The estimate-pixel values PVE(G1), . . . , PVE(Gn) respectively indicate magnitudes of the preset pixel signals PS2(G1), . . . , PS2(Gn) output by the amplification circuit 62 before the gain gG is set. The estimate-pixel values PVE(B1), . . . , PVE(Bn) respectively indicate magnitudes of the preset pixel signals PS2(B1), . . . , PS2(Bn) output by the amplification circuit 62 before the gain gB is set.

The display control unit 15 causes the display to present a frame image corresponding to the estimated estimate-pixel values PVE(R1), . . . , PVE(Rn), PVE(G1), . . . , PVE(Gn), and PVE(B1), . . . , PVE(Bn). Such a feature makes it possible to present on the display a natural moving image having an RGB ratio reflecting the actual color of the human body HB before the white balance 33 is corrected.

The present disclosure shall not be limited to the above-described embodiments, and may be replaced with a configuration substantially the same as a configuration having the same advantageous effects as, or a configuration capable of achieving the same object as, the configurations described in the above-described embodiments.

What is claimed is:

1. A biological signal obtaining device, comprising:
an imaging unit configured to perform imaging in accordance with an imaging condition, and to output pixel values of a plurality of pixels in each of channels that are included in two or more channels each corresponding to one of two or more wavelengths different from one another;
a representative value calculating unit configured to calculate a representative value of each of the channels from pixel values of a plurality of intra-region pixels in a region showing a biological subject, the pixel values being included in the pixel values of the plurality of pixels;
a control unit configured to set the imaging condition so that the representative value of each of the channels is greater than, or equal to, a set value of each of the channels; and
a biological signal calculating unit configured to calculate a biological signal from representative values of the two or more channels.

2. The biological signal obtaining device according to claim 1,
wherein the set value of each of the channels is greater than, or equal to, a center value within a range of the pixel values of the plurality of pixels.

3. The biological signal obtaining device according to claim 1,
wherein the biological signal is a signal indicating a pulse wave.

4. The biological signal obtaining device according to claim 1,
wherein the imaging condition includes a gain of each of the channels,
the pixel values of the plurality of pixels have a first bit length, and
the imaging unit includes:
a pixel signal output unit configured to output pixel signals of the plurality of pixels;
a quantization unit configured to quantize the pixel signals of the plurality of pixels in order to obtain pre-amplified pixel values of the plurality of pixels, the pre-amplified pixel values having a second bit length longer than the first bit length;
an amplification unit configured to amplify the pre-amplified pixel values of the plurality of pixels with a gain of each of the channels in order to obtain amplified pixel values of the plurality of pixels, the amplified pixel values having the second bit length; and
a conversion unit configured to convert the amplified pixel values of the plurality of pixels into the pixel values of the plurality of pixels.

5. The biological signal obtaining device according to claim 4,
wherein calculating the representative value of each of the channels from the pixel values of the plurality of intra-region pixels includes: estimating estimate-pixel values of the plurality of intra-region pixels from the gain of each of the channels and set pixel values of the plurality of intra-region pixels; and calculating the representative value of each of the channels from the estimate-pixel values of the plurality of intra-region pixels, the estimate-pixel values being estimate values of pre-amplified pixel values of the plurality of intra-region pixels and having the second bit length, and the set pixel values being output by the imaging unit after the gain of each of the channels has been set.

6. The biological signal obtaining device according to claim 4, further comprising
a display control unit configured to estimate estimate-pixel values of the plurality of pixels from the gain of each of the channels and set pixel values of the plurality of pixels and to cause a display to present an image based on the estimate-pixel values of the plurality of pixels, the estimate-pixel values being estimate values of pre-amplified pixel values of the plurality of pixels, and the set pixel values being output by the imaging unit after the gain of each of the channels has been set.

7. The biological signal obtaining device according to claim 1,
wherein the imaging condition includes a gain of each of the channels,
the imaging unit includes:
a pixel signal output unit configured to output pixel signals of the plurality of pixels;
an amplification circuit configured to amplify the pixel signals of the plurality of pixels with the gain of each of the channels in order to obtain amplified pixel signals of the plurality of pixels; and
a quantization unit configured to quantize the amplified pixel signals of the plurality of pixels in order to obtain the pixel values of the plurality of pixels.

8. The biological signal obtaining device according to claim 7,
wherein the pixel values of the plurality of pixels have a first bit length, and
calculating the representative value of each of the channels from the pixel values of the plurality of intra-region pixels includes: estimating estimate-pixel values of the plurality of intra-region pixels from the gain of each of the channels and set pixel values of the plurality of intra-region pixels; and calculating the representative value of each of the channels from the estimate-pixel values of the plurality of intra-region pixels, the estimate-pixel values indicating magnitudes of preset pixel signals of the plurality of intra-region pixels and having a second bit length longer than the first bit length, the preset pixel signals being output by the amplification circuit before the gain of each of the channels is set, and the set pixel values being output by the imaging unit after the gain of each of the channels has been set.

9. The biological signal obtaining device according to claim 7, further comprising
a display control unit configured to estimate estimate-pixel values of the plurality of pixels from the gain of each of the channels and set pixel values of the plurality of pixels and to cause a display to present an image based on the estimate-pixel values of the plurality of pixels, the estimate-pixel values indicating magnitudes of preset pixel signals of the plurality of pixels, the preset pixel signals being output by the amplification circuit before the gain of each of the channels is set, and the set pixel values being output by the imaging unit after the gain of each of the channels has been set.

10. The biological signal obtaining device according to claim 4,
wherein setting the imaging condition includes changing gains of the two or more channels from a reference gain of the two or more channels.

11. The biological signal obtaining device according to claim 4,
wherein the two or more channels includes a first channel and a second channel, and
setting the imaging condition includes setting a ratio of a gain of the second channel to a gain of the first channel, setting the gain of the first channel as a reference gain of the first channel, and setting the gain of the second channel as a product of the reference gain and the ratio.

12. The biological signal obtaining device according to claim 1,
wherein the control unit sets the imaging condition so that the representative values of the two or more channels approximate to one another.

13. The biological signal obtaining device according to claim 12,
wherein the imaging condition includes a gain of each of the channels, and
setting the imaging condition so that the representative values of the two or more channels approximate to one another includes increasing the gain of the channel as a preset representative value of the channel becomes smaller, the preset representative value being calculated by the representative value calculating unit before a gain of a channel included in the two or more channels is set.

14. The biological signal obtaining device according to claim 4,
wherein the two or more channels include a red channel, a green channel, and a blue channel, and
setting the imaging condition includes: setting a gain of the red channel to 1; setting a gain of the green channel to a ratio of a preset representative value of the red channel to a preset representative value of the green channel, the preset representative value of the red channel being calculated by the representative value calculating unit before the gain of the red channel is set, and the preset representative value of the green channel being calculated by the representative value calculating unit before the gain of the green channel is set; and setting a gain of the blue channel to a ratio of the preset representative value of the red channel to a preset representative value of the blue channel, the preset representative value of the blue channel being calculated by the representative value calculating unit before the gain of the blue channel is set.

15. The biological signal obtaining device according to claim 1,
wherein the control unit sets the imaging condition so that the representative value of each of the channels is smaller than, or equal to, an upper limit set value of each of the channels.

16. The biological signal obtaining device according to claim 15,
wherein the upper limit set value of each of the channels is smaller than an upper limit value within a range of the pixel values of the plurality of pixels.

17. The biological signal obtaining device according to claim 1,
wherein the representative value calculating unit is capable of calculating, as the representative value of each of the channels: a first representative value of each of the channels;
and a second representative value of each of the channels, the second representative value being different from the first representative value,
setting the imaging condition so that the representative value of each of the channels is greater than, or equal to, a set value of each of the channels includes setting the imaging condition so that the first representative value of each of the channels is greater than, or equal to, the set value of each of the channels, and
calculating the biological signal from the representative values of the two or more channels includes calculating the biological signal from second representative values, of the two or more channels, including the second representative value.

18. A biological signal obtaining method, comprising:
imaging in accordance with an imaging condition, and outputting pixel values of a plurality of pixels in each of channels included in two or more channels each corresponding to one of two or more wavelengths different from one another;
calculating a representative value of each of the channels from pixel values of a plurality of intra-region pixels in a region showing a biological subject, the pixel values being included in the pixel values of the plurality of pixels;
setting the imaging condition so that the representative value of each of the channels is greater than, or equal to, a set value of each of the channels; and
calculating a biological signal from representative values of the two or more channels.

19. A computer-readable recording medium containing a program to cause a computer to execute:
imaging, by an imaging unit, in accordance with an imaging condition, and outputting, by the imaging unit, pixel values of a plurality of pixels in each of channels included in two or more channels each corresponding to one of two or more wavelengths different from one another;
calculating a representative value of each of the channels from pixel values of a plurality of intra-region pixels in a region showing a biological subject, the pixel values being included in the pixel values of the plurality of pixels;
setting the imaging condition so that the representative value of each of the channels is greater than, or equal to, a set value of each of the channels; and
calculating a biological signal from representative values of the two or more channels.
